(19) United States
(12) Patent Application Publication
(10) Pub. No.: US 2026/0325827 A1
(43) Pub. Date: Oct. 22, 2026

(54) LINE DEVICE FOR CONDUCTING A BLOOD FLOW FOR A HEART SUPPORT SYSTEM, AND PRODUCTION AND ASSEMBLY METHOD

(71) Applicant: KARDION GMBH, Stuttgart (DE)

(72) Inventors: Ingo Stotz, Ditzingen (DE); Hardy Baumbach, Stuttgart (DE); Inga Schellenberg, Stuttgart (DE); David Minzenmay, Stuttgart (DE)

(21) Appl. No.: 19/724,558

(22) Filed: Jun. 29, 2026

Related U.S. Application Data
(63) Continuation of application No. 17/057,355, filed on May 30, 2019, now Pat. No. 12,667,714.

(30) Foreign Application Priority Data
May 30, 2018 (DE) 102018208536.6

(51) Int. Cl. A61M 60/859; A61M 60/139; A61M 60/148; A61M 60/178; A61M 60/216

(52) U.S. Cl. CPC A61M 60/859; A61M 60/139; A61M 60/148; A61M 60/178; A61M 60/216

(57) ABSTRACT

The invention relates to a line device (105) for conducting a blood flow for a heart support system. The line device (105) has a main part (205), and the main part (205) has a first attachment section (210) at a first end for attaching the line device (105) to a head unit of the heart support system and a second attachment section (215) at a second end for attaching the line device (105) to an outlet unit of the heart support system (100). The attachment sections (210, 215) are shaped so as to be connectable in a form-fitting and/or force-fitting manner. The main part (205) has a structured section (220) with at least one stiffening recess (225) between the attachment sections (210, 215), the at least one stiffening recess (225) being shaped so as to change the stiffness of the main part (205).

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is a continuation of U.S. patent application number 17/057355, entitled LINE DEVICE FOR CONDUCTING A BLOOD FLOW FOR A HEART SUPPORT SYSTEM, AND PRODUCTION AND ASSEMBLY METHOD, and filed May 18, 2021, which is the U.S. National Phase of international application number PCT/EP2019/064136, entitled LINE DEVICE FOR CONDUCTING A BLOOD FLOW FOR A HEART SUPPORT SYSTEM, AND PRODUCTION AND ASSEMBLY METHOD, and filed May 30, 2019, which claims the benefit of priority to German patent application number 102018208536.6, entitled LINE DEVICE FOR CONDUCTING A BLOOD FLOW FOR A HEART SUPPORT SYSTEM, AND PRODUCTION AND ASSEMBLY METHOD, and filed May 30, 2018. Each of the above-referenced application is hereby incorporated by reference herein in its entirety for all purposes and forms a part of this specification.

### BACKGROUND

### Field

The invention relates to cardiac support systems, devices, and methods.

### Description of the Related Art

Cardiac support systems, in particular left ventricular support systems, can be differentiated in terms of their position on the heart and their access to the blood stream. Long-term support systems can be positioned at the apex of the heart (transapically) and bypass the left ventricle by pumping blood from the apex of the heart through a hose directly into the aorta. Another type of access can be used in particular for short-term support of the heart. For example, the cardiac support system can be a ventricular support system as a bridging measure; as a bridge to a transplant (bridge to decision, bridge to transplant). The natural aortic valve can be used to create a connection between the pump inlet and the pump outlet. With such an arrangement of the cardiac support system, the aorta can be used as an access route (transaortic) in the context of a minimally invasive surgical procedure and a sternotomy can be avoided.

### SUMMARY

An underlying object of the invention is to specify an improved line device for a cardiac support system, in particular with long-lasting connection reliability and suitable flexibility, as well as a method for the production and assembly of said device.

With that in mind, the approach presented here proposes a line device for conducting a blood flow for a cardiac support system, a method for producing a line device and a method for assembling a cardiac support system according to the main claims. Advantageous further developments and improvements of the device specified in the independent claim are possible using the measures listed in the dependent claims.

This approach presents a line device for conducting a blood flow for a cardiac support system, for example a left ventricular cardiac support system. The line device can be used as a flow channel, in which the blood flow can be conducted from the pump inlet in a left ventricle to the pump outlet inside an aorta. A head unit of the cardiac support system can be attached to the line device at one end, and a further component of the cardiac support system, for example an impeller housing, can be attached at another end. The attachment can be realized by means of a form-locking connection. Additionally or alternatively, the connection can also be realized in a force-locking manner. The line device can advantageously be configured to enable a transfemoral surgical procedure (access via the groin) to implant the cardiac support system. This can in particular be achieved via the ratio of flexibility and stiffness of the line device.

A line device for conducting a blood flow for a cardiac support system is presented. The line device comprises a main body, wherein the main body comprises a proximal (first) attachment section at a first end, upstream, for attaching the line device to a head unit of the cardiac support system and comprises a distal (second) attachment section at a second end, downstream, for attaching the line device to an outlet unit of the cardiac support system. The attachment sections are formed to be connectable in a form-locking and additionally or alternatively force-locking manner. The main body further comprises a structural section having at least one stiffening recess between the attachment sections, wherein the at least one stiffening recess is formed to change the stiffness of the main body.

The line device can be made of a biocompatible material and can be understood to be a flow channel for flexibly connecting components of a cardiac support system for conducting a blood flow between the ventricle and the blood vessel. The line device can be used as a suction hose for the cardiac support system to initiate blood flow and to conduct it to an outlet section of the cardiac support system. The cardiac support system can, for example, be understood to be a left ventricular support system (LVAD, left ventricular assist device) or other ventricular support system (VAD, ventricular assist device). The main body of the line device can be configured as a hollow cylinder, for example, and substantially have a tubular geometry. The proximal attachment section at a first end of the main body can be understood to be a first attachment section and can be disposed in the left ventricle, for example, as a left ventricular support system when the cardiac support system is implanted. The distal attachment section at a second end of the main body can be understood to be a second attachment section and can be disposed in the aorta, for example, when the left ventricular support system is implanted. A head unit of the cardiac support system, for example a sensor assembly, can be attached to the proximal attachment section. The structural section can, for example, be a main body section having a predefined sectional contour for changing the stiffness of the line device, for example to enable the use of the line device during a transfemoral surgical procedure. The stiffness can be changed via the configuration of the sectional contour of the structural section, in particular a number and shape of the at least one stiffening recess. The at least one stiffening recess can have a spiral shape or a wave shape, for example. The inlet section can, for example, be realized by a multi-part window in the main body. The inlet section can be configured to enable the initiation of blood flow into the main body of the line device.

According to one embodiment, at least one of the attachment sections can comprise at least one connection element for form-locking connection. The connection element can be, for example, a recess or an elevation, for example a knob, or a threaded element. The connection element as a recess or as a knob can, for example, be round, oval, triangular, polygonal or star-shaped. It is also possible for each of the attachment sections to comprise a plurality of connection elements, wherein the connection elements can also be formed differently. Depending on the shape of the attachment sections, the correspondingly formed engaging or gripping connection partner of the connection element can be realized for connecting the line device to the head unit and additionally or alternatively to the outlet unit of the cardiac support system.

According to a particularly advantageous embodiment, the main body can comprise an inlet section between the structural section and the proximal or first attachment section, wherein the inlet section is formed to allow initiation of the blood flow. This makes it possible to achieve a very advantageous guidance of a blood flow, i.e. make advantageous use of the structural section.

Furthermore, according to one embodiment, at least one portion of the main body can be made of a shape memory material according to an embodiment. The shape memory material can be a biocompatible shape memory polymer, or a biocompatible shape memory alloy, such as Nitinol for example. It is also possible for the entire main body to be made of the shape memory material. The use of Nitinol as a shape memory material is advantageous, because the Nitinol material is a proven material in the field of medicine, in particular in the field of cardiovascular medicine, for example for heart valve prostheses, stents and vascular prostheses, and, due to its biocompatibility and the shape memory property, makes it possible to realize even complex structures in a small installation space.

According to one embodiment, the line device can also comprise a cable groove. The cable groove can be configured to guide a cable along the main body. The cable groove can in particular be configured to extend helically around the structural section. The cable can be a cable for signal transmission, for example, and additionally or alternatively for power transmission. If, for example, the head unit attached to the line device comprises a sensor, the cable held by the cable groove can be configured to take sensor data from the head unit at a pump tip inside the heart valve and transmit it to a control device. The cable groove can advantageously help prevent the cable from breaking during operational movements of the cardiac support system.

According to one embodiment, the structural section of the line device can extend over at least half of the main body. This is advantageous with regard to implantation of the line device, in particular in the case of transfemoral access, for enabling a predefined ratio of flexibility and stiffness of the line device via the configuration of the structural section. The flexibility of the line device can be advantageous, for example when being pushed through an aortic arch, and the stiffness can advantageously prevent the line device from bending when being pushed through a blood vessel.

The structural section can also comprise a plurality of slotted holes as the at least one stiffening recess. The longitudinal holes can be evenly spaced and be configured to extend obliquely transverse to the longitudinal axis of the structural section. The plurality of slotted holes can also be configured to extend helically around the structural section. The use of a plurality of slotted holes as the at least one stiffening recess is advantageous, for example, for adjusting the flexibility of the line device via the spacing of the individual slotted holes.

According to one embodiment, the line device can also comprise a sealing layer. The sealing layer can be disposed on the structural section and configured to seal the at least one stiffening recess in a fluid-tight manner. The fluid-tight sealing of the at least one stiffening recess by means of the sealing layer is advantageous for conducting the blood flow to be able to conduct the blood flow to the outlet unit without loss. The sealing section can be realized, for example, by potting or overmolding the structural section with a flexible plastic, such as polyurethane or silicone.

According to one embodiment, the main body can also comprise a bending point, wherein the bending point is disposed between the inlet section and the distal attachment section. The main body can have a first longitudinal axis between the distal attachment section and the bending point, and the main body can have a second longitudinal axis oblique to the first longitudinal axis between the bending point and the proximal attachment section, whereby the main body is curved in a region of the bending point. The bending point can be configured to give the main body a curved shape corresponding to the human anatomy, for example, to enable a positioning of the inlet section in the center of a ventricle to advantageously avoid suctioning of the inlet unit to a ventricular wall.

According to one embodiment, an inner diameter of the main body can furthermore change from the proximal attachment section to the distal attachment section. For example, a cross-section of the inner diameter can taper in the direction of the distal attachment section. The change in the inner diameter of the main body can advantageously improve the flow properties of the initiated blood flow.

According to one embodiment, the inlet section can comprise at least one inlet opening cut into the main body. The inlet opening can be rectangular, for example, or shaped as a rectangle with a circular arc in the direction of the structural section. The inlet section can also comprise a plurality of inlet openings, for example three inlet openings. In this case, the inlet openings can be evenly spaced, for example, whereby a narrow web can connect the proximal attachment section to the structural section between two adjacent inlet openings, for example. By forming at least one inlet opening, which can be cut into the main body, there is advantageously no need for an additional structural element for initiating the blood flow, which is advantageous with regard to a compact design.

According to one embodiment, the distal attachment section can comprise a lead-out opening for leading out a guide wire for positioning the cardiac support system. The guide wire can be inserted at the proximal attachment section, for example, and led out through the lead-out opening. This is advantageous to enable a positioning of the line device along the guide wire and also to prevent damage to the guide wire or pump components.

A cardiac support system is presented as well. The cardiac support system can comprise a head unit, an outlet unit and an embodiment of the aforementioned line device. The line device can be disposed between the head unit and the outlet unit and connected to the head unit and the outlet unit.

A method for producing a line device for conducting a blood flow for a cardiac support system is presented as well. The method includes the following steps:
forming a main body from a semi-finished product made of a shape memory material, wherein the main body comprises a proximal attachment section at a first end for attaching the line device to a head unit of the cardiac support system and comprises a distal attachment section at a second end for attaching the line device to an outlet unit of the cardiac support system, wherein the attachment sections are formed to be connectable in a form-locking and/or force-locking manner, wherein the main body comprises a structural section having at least one stiffening recess between the attachment sections, wherein the at least one stiffening recess is formed to change the stiffness of the main body, and
heat treating the formed main body in order to emboss a predetermined shape on the main body.

An embodiment of the aforementioned line device can advantageously be produced by carrying out the aforementioned method.

A method for assembling an embodiment of the aforementioned cardiac support system is presented as well. The method includes the following step:
producing a form-locking and/or force-locking connection of the head unit and the outlet unit to the line device to assemble the cardiac support system.

### BRIEF DESCRIPTION OF THE DRAWINGS

Design examples of the invention presented here are shown schematically in the drawings and explained in more detail in the following description. The figures show:

FIG. 1 a perspective illustration of a cardiac support system with a line device for conducting a blood flow;

FIG. 2 a side view of a line device for conducting a blood flow for a cardiac support system;

FIG. 3 a side view of a further line device for conducting a blood flow;

FIG. 4 a broken off perspective illustration of a distal attachment section of a line device;

FIG. 5 a broken off perspective illustration of an impeller housing of a cardiac support system;

FIG. 6 a broken off perspective view of a distal attachment section of a line device connected to the impeller housing;

FIG. 7 a schematic illustration of a connection element;

FIG. 8 a broken off perspective illustration of a proximal attachment section of a line device;

FIG. 9 a cut-out perspective illustration of a distal attachment section of a line device;

FIG. 10 a perspective illustration of a bending point of a line device;

FIG. 11 a schematic side view of a line device;

FIG. 12 a perspective, partially cross-sectional illustration of a part of a cardiac support system;

FIG. 13 a perspective illustration of a line device;

FIG. 14 a perspective illustration of a part of a cardiac support system;

FIG. 15 a perspective illustration of a structural section of a line device;

FIG. 16 a perspective illustration of a line device;

FIG. 17 a perspective illustration of a line device;

FIG. 18 a schematic illustration of a part of a line device;

FIG. 19 a cut-out enlargement of the line device of FIG. 18;

FIG. 20 a flow diagram of a production method; and

FIG. 21 a flow diagram of an assembly method.

### DETAILED DESCRIPTION

In the following description of favorable design examples of the present invention, the same or similar reference signs are used for the elements shown in the various figures, which have a similar effect, whereby a repeated description of these elements is omitted.

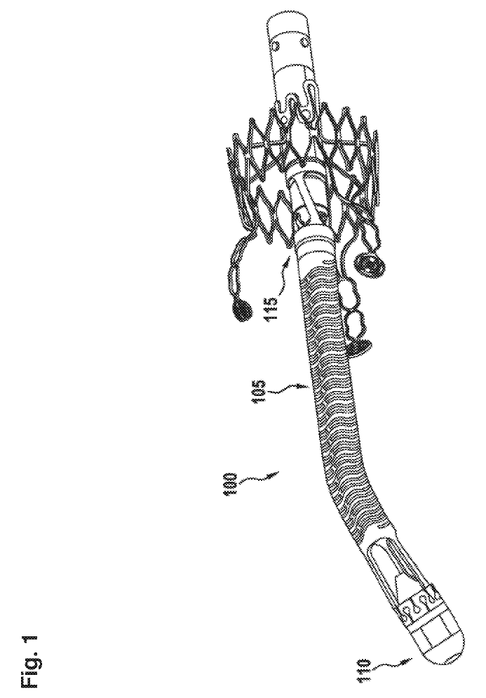

FIG. 1 shows a cardiac support system 100 with a hose-like line device 105 for conducting a blood flow according to a design example. The figure shows a view of the cardiac support system 100 as an overall system which, as an example, is configured here as a left ventricular support system. The cardiac support system 100 comprises a head unit 110, an outlet unit 115 and the line device 105. The line device 105 is disposed between the head unit 110 and the outlet unit 115 and connected to the head unit 110 and the outlet unit 115. The line device 105 can also be referred to as a suction hose, which, in the implanted state of the cardiac support system 100, connects a pump inlet inside a ventricle to an outlet inside the aorta.

The cardiac support system 100 has a cylindrical, elongated structure with a substantially constant outer diameter and rounded, tapering ends for easy placement in a blood vessel, such as the aorta, by means of a catheter. The cardiac support system 100 is furthermore bent in the direction of the head unit 110, whereby the bend is configured, for example, as an obtuse angle with respect to a longitudinal axis of the cardiac support system 100.

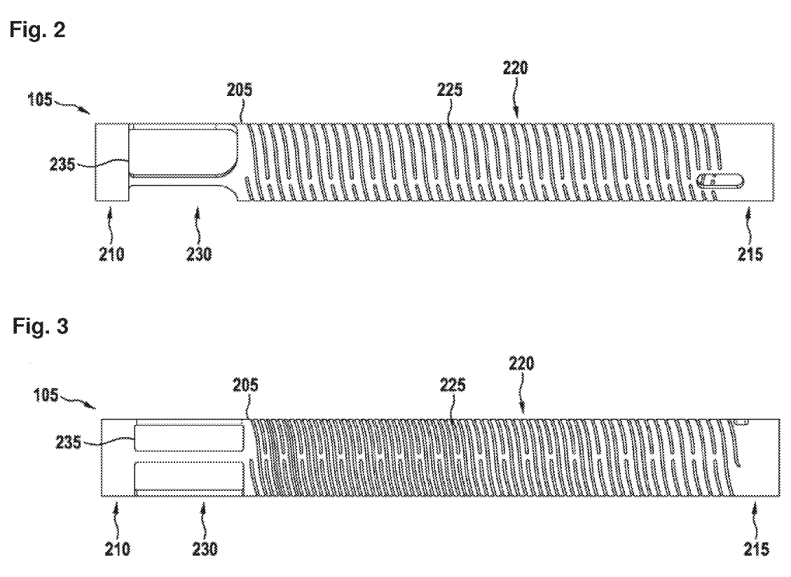

FIG. 2 shows a schematic illustration of a line device 105 for conducting a blood flow for a cardiac support system according to a design example in a side view. The line device 105 can also be referred to as a laser-cut suction hose. The line device 105 comprises a main body 205. The main body 205 is tube-shaped for example. The main body 205 comprises a proximal attachment section 210 at a first end for attaching the line device 105 to a head unit of the cardiac support system and comprises a distal attachment section 215 at a second end for attaching the line device 105 to an outlet unit of the cardiac support system. The attachment sections 210 and 215 are formed to be connectable in a form-locking and/or force-locking manner. The main body 105 further comprises a structural section 220 having at least one stiffening recess 225 between the attachment sections 210, 215, wherein the at least one stiffening recess 225 is formed to change the stiffness of the main body 205. The main body 205 also comprises an inlet section 230 between the structural section 220 and the proximal attachment section 205, wherein the inlet section 230 is formed to allow initiation of the blood flow.

The structural section 220 extends over at least half of the main body 205 as shown here according to design examples. According to the design example shown here, the structural section 220 comprises a plurality of slotted holes as the stiffening recess 225. The stiffening recesses 225 extend over the entire structural section 220, for example, and are disposed helically around said section.

According to the design example shown here, the inlet section 230 comprises at least one inlet opening 235 cut into the main body 205. As an example, the inlet opening 235 here is realized as a multi-part window. There is therefore no need for an additional structural element for the inflow of blood initiated by the inlet section 230. According to the design example shown here, the inlet section 230 comprises three rectangular inlet openings 235, which are rounded in the direction of the structural section 220 in the form of a circular arc.

FIG. 3 shows a schematic illustration of a line device 105 for conducting a blood flow for a cardiac support system according to a design example. The depicted line device 105 is the same as or similar to the line device of FIG. 2 described above, except for the distance between the plurality of slotted holes as the stiffening recess 225. The structural section 220 shown here has a finer sectional contour than the coarser sectional contour shown in FIG. 2. Adjacent slotted holes are therefore closer to one another here in FIG. 3. The ratio of flexibility and stiffness of the line device 105 can be set via the laser-cut structure, for example. This is of great importance with respect to transfemoral access during a surgical procedure to implant the cardiac support system with the line device 105, because the support system, also referred to as the pump, has to be flexible enough to be pushed through the aortic arch and also requires a certain amount of stiffness to be pushed through the narrow blood vessels in axial direction by an acting force without bending.

Another difference to the already described design example of the line device 105 is the configuration of the inlet section 230. In FIG. 3, the inlet section 230 also comprises three inlet openings 235. The inlet openings 235 here are rectangular in shape, as an example, whereby the sides of each inlet opening 235 that are opposite to one another along the longitudinal axis of the line device 105 are longer than the sides that are opposite to another transverse to the longitudinal axis. The proximal attachment section 210 and the distal attachment section 215 correspond to the design example described in FIG. 2 above.

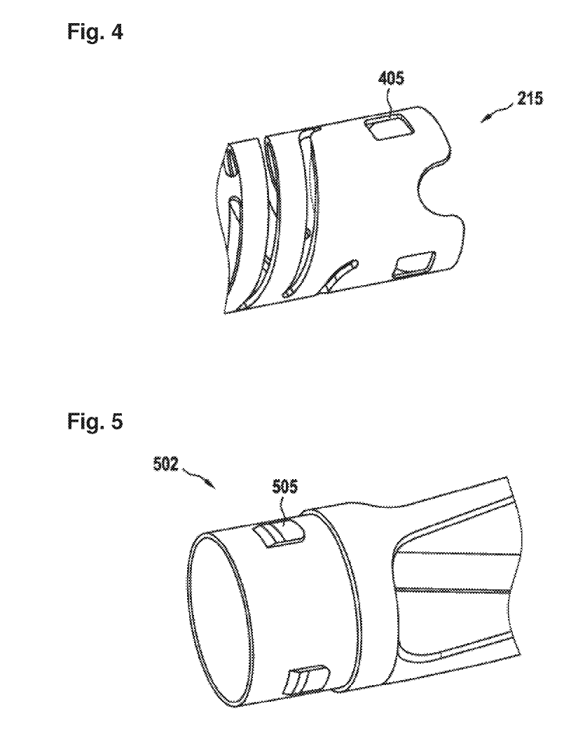

FIG. 4 shows a schematic illustration of a distal attachment section 215 of a line device according to a design example. The line device is the same as or similar to the line device of one of the figures described above. The distal attachment section 215 comprises at least one connection element 405 for form-locking connection. The connection element 405 is shown here as an example as a rectangular recess. The connection element 405 can alternatively also be realized as an elevation, such as the connection element shown in the following FIG. 5. The proximal attachment section optionally comprises a corresponding or similar connection element 405 as the connection element 405 shown here for form-locking connection. The connection element 405 is implemented with the aid of a laser-cut contour, for example, to realize the connection in a fatigue endurable and reliable manner using a key-and-lock principle. Such a laser-cut distal attachment section 215 based on the key-and-lock principle is shown here as an example. The connection element 405, which is rectangular here, can also have a different geometric shape. For example, the connection element 405 can be realized as a circle, as an oval, as a triangle, as a polygon or as a star. If the connection element 405 has an elevation, the elevation can have a flattened section as described with reference to FIG. 7.

FIG. 5 shows a schematic illustration of an impeller housing 502 of a cardiac support system. The impeller housing 502 can correspond to the outlet unit of the cardiac support system described with reference to FIG. 1 or be part of the outlet unit. In a side view of the impeller housing 502, a rectangular connection element 505 is shown as a knob on the impeller housing 505. The counterpart, and therefore the connection partner of the recess described with reference to FIG. 4, is shown here as an example as the connection element. The connection element shown here has a flattened side to facilitate the assembly process, i.e. the form-locking connection of the impeller housing 505 to the line device. The connection element for the form-locking connection of one of the attachment sections can be configured like the connection element 505 shown here and realized on one of the attachment sections.

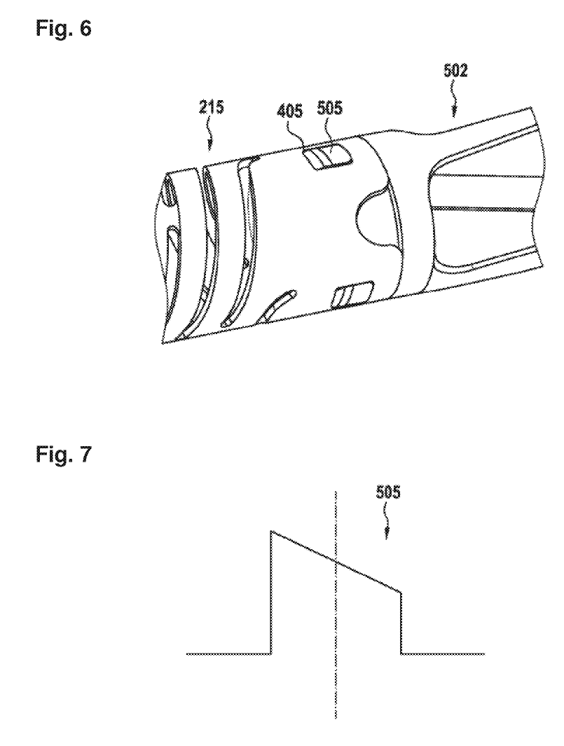

FIG. 6 shows a schematic illustration of a distal attachment section 215 of a line device according to a design example connected to the impeller housing 502. The distal attachment section 215, as described with reference to FIG. 4 above, and the impeller housing, as described with reference to FIG. 5, are shown in the connected, i.e. assembled state. An assembled form-locking attachment point between the line device in the form of the distal attachment section 215 and the adjacent structural element, the impeller housing 502, is accordingly shown here as an example. As an example, the connection element 505 here has a projecting shape with a flattened side to facilitate the form-locking connection when gripping the connection element 405. The distal attachment section 215 is configured as a part of the line device, which is realized as a Nitinol tube as an example.

FIG. 7 shows a schematic illustration of a connection element 505 according to a design example. The figure shows a cross-section of the connection element 505, which here has a flattened section. The shown connection element 505 is the same as or similar to the connection element as described with reference to FIG. 5 and FIG. 6 above.

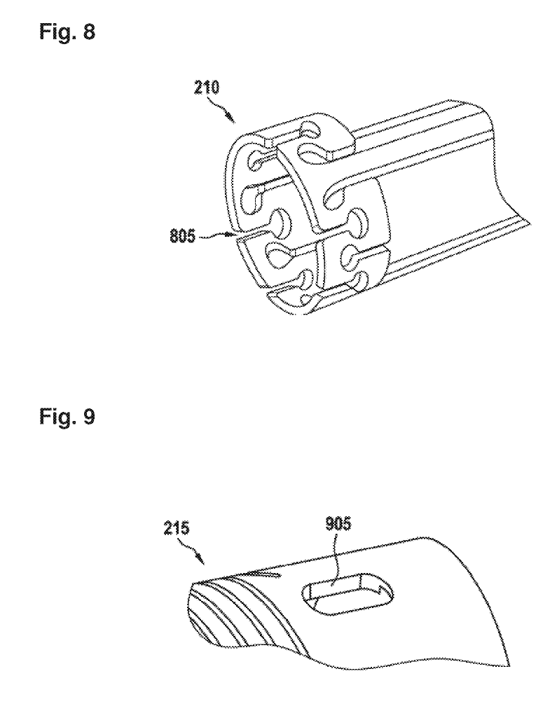

FIG. 8 shows a schematic illustration of a proximal attachment section 210 of a line device according to a design example. The line device is the same as or similar to the line device of one of the figures described above. The figure shows a laser cut at the proximal end of the line device as the proximal attachment section 210. The shape of the proximal attachment section 210 shown here allows widening and thus a force-locking press fit. According to the design example shown here, the head unit of the cardiac support system is connected to the proximal attachment section 210 in a force-locking manner. As an example, the proximal attachment section 210 comprises labyrinth recesses 805 to enable widening of the proximal attachment section 210 and to be able to create a force-locking connection to a corresponding counterpart. The labyrinth recesses 805 are realized circumferentially on the attachment section. Along the longitudinal axis of the line device, each labyrinth recess 805 has a slit-shaped opening that widens in a circular manner, whereby the slit-shaped openings of two adjacent labyrinth recesses 805 are opposite to one another.

FIG. 9 shows a schematic illustration of a distal attachment section 215 of a line device according to a design example. The line device is the same as or similar to the line device of one of the figures described above. The distal attachment section 215 here comprises a lead-out opening 905 for leading out a guide wire for positioning the cardiac support system. The lead-out opening 905 has an elongated shape for example. The elongated shape of the lead-out opening 905 allows the guide wire to be led out and forwarded at a shallow angle. The guide wire is inserted through an opening in the proximal tip of the pump, i.e. the cardiac support system, and passed out through the lead-out opening 905. This allows the cardiac support system to be positioned along the guide wire without the guide wire touching or damaging the impeller.

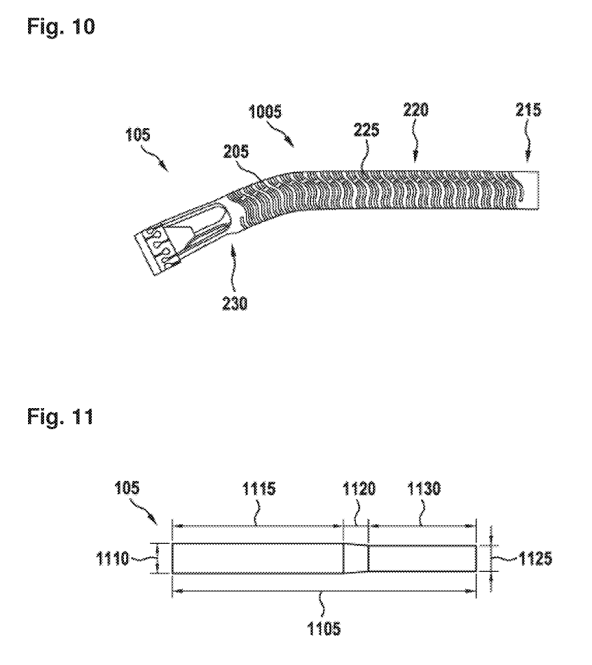

FIG. 10 shows a schematic illustration of a bending point 1005 of a line device 105 according to a design example. The line device 105 is the same as or similar to the line device of one of the figures described above. The main body 205 comprises the bending point 1005, wherein the bending point 1005 is disposed between the inlet section 230 and the distal attachment section 215. As an example, the bending point 1005 is configured here to enable the positioning of the inlet section 230 in the center of the ventricle when implanting the cardiac support system with the line device 105. The line device 105 is bent at an obtuse angle along a first longitudinal axis by the bending point 1005. The line device accordingly has a first longitudinal axis, and a second longitudinal axis extends obliquely to the first longitudinal axis after the bending point 1005 to shape the main body 205 in a curved manner in the direction of the inlet section 230.

The main body 205 comprises a first longitudinal axis between the distal attachment section 215 and the bending point 1005. Between the bending point 1005 and the proximal attachment section 210, the main body 205 has a second longitudinal axis at an angle to the first longitudinal axis. The second longitudinal axis is at an obtuse angle to the first longitudinal axis. The main body 205 is curved in the region of the bending point 1005.

According to a design example, at least one portion of the main body 205 can be made of a shape memory material. In the design example shown here, the entire main body 205 is made of one material, for example Nitinol. Due to the shape memory properties of the Nitinol, a curved shape corresponding to the anatomy can be imparted after laser cutting the main body 205 from a Nitinol tube to be able to position the inlet section 230 in the center of the ventricle in order to avoid suctioning of the hose opening of the line device 105, i.e. the inlet section 230, to a ventricular wall. The Nitinol material is a proven material in the field of medicine, in particular in the field of cardiovascular medicine, for example for heart valve prostheses, stents and vascular prostheses. Nitinol combines the advantages of biocompatibility and the shape memory property that makes it possible to realize even complex structures in a small installation space. In the design example shown here, the main body 205 of the line device 105 is made of an elastic material, of Nitinol. A tubular geometry is a suitable semi-finished product for the machining of the main body 205. The semi-finished product can have a wall thickness of 0.25 to 0.5 millimeters, for example. The contour of the main body 205 shown here is realized by methods for material removal, for example laser cutting, by removing pipe volume at some locations. Punching and erosion methods or machining are alternatively possible as well. It is thus possible to impart a helix or wave shape into the tube of the main body 205. The spacing of the individual sections of the structural section 220 can also be varied. The distance between the individual sections can be 0.5 millimeters, for example. The dimensions of the stiffening recess 225 and the spacing of the stiffening recesses 225 changes the flexibility and stiffness of the line device 105. The larger the stiffening recess 225 and the smaller the spacing of the cuts, the more flexible the tube of the line device 105 becomes. The laser-cut contour can be given a specific shape as part of a heat treatment, for example, (e.g. with a temperature T of at least 500 degrees Celsius), e.g. with a bend of the main body 205 in the form of the bending point, or with different diameters, as described with reference to the following FIG. 11. The embossing process of the shape of the main body 205 is a plastic deformation without the occurrence of material failure.

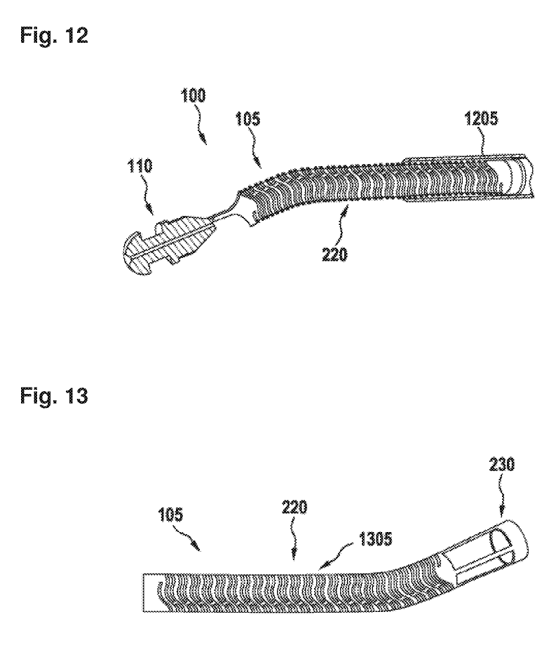

FIG. 11 shows a schematic illustration of a line device 105 according to a design example. The line device 105 is the same as or similar to the line device of one of the figures described above. The figure shows a change in an inner diameter of the main body of the line device 105, which is illustrated using exemplary dimensions of the line device 105. The main body of the line device has a length of 62 millimeters illustrated by the label 1105. According to the design example shown here, an inner diameter of the main body changes from the proximal attachment section to the distal attachment section. The cross-section of the proximal attachment section has a diameter of 6 millimeters, as indicated by the label 1110. The label 1115 indicates a length of 35 millimeters of a section of the main body with the proximal attachment section having a continuous inner diameter of 6 millimeters. In the section marked by the label 1120, which has a length of 5 millimeters and adjoins the section of the label 1115, the inner diameter of the line device 105 tapers from 6 millimeters to 5.49 millimeters, as indicated by the label 1125. Over the remaining length of 22 millimeters of the main body of the line device 105 shown here, indicated by the label 1130, the inner diameter remains constant at 5.49 millimeters. The change in the inner diameter of the main body shown here can improve the flow properties of the blood flow. For this purpose, different diameters can be imposed on the line device 105 in axial direction as shown here. The inner diameter can in particular be larger in the region with the proximal attachment section indicated as an example by the label 1115 than in the region with the distal attachment section indicated by the label 1130. In the region corresponding to the label 1115, for example, a larger installation space can be available than in the region indicated by the label 1130, for example if the region 1130 is enclosed by a further structural element, for example a sleeve, during implantation of the cardiac support system for insertion of the cardiac support system into a catheter, as shown in the following FIG. 12.

FIG. 12 shows a schematic illustration of a part of a cardiac support system 100 according to a design example. The cardiac support system 100 is the same as or similar to the cardiac support system of FIG. 1. The figure shows a longitudinal section of the part of the cardiac support system 100 in the assembled state of the cardiac support system 100, which comprises the head unit 110 and the line device 105. The cardiac support system 100 also comprises a sleeve 1205. The sleeve 1205 encloses a part of the cardiac support system opposite to the head unit 110, wherein the sleeve 1205 encloses about half of the structural section 220 and the distal attachment section of the line device 105. The sleeve 1205 is shown here only allusively to illustrate an insertion situation of the cardiac support system with a line device 105 in terms of the configuration of the line device 105. Due to the arrangement of the sleeve 1205, the region of the line device 105 not enclosed by the sleeve 1205 has a larger usable installation space than the region enclosed by the sleeve 1205.

FIG. 13 shows a schematic illustration of a line device 105 according to a design example. The line device 105 is the same as or similar to the line device of one of the figures described above. The figure shows a plan view onto the line device 105. According to the design example shown here, the line device comprises a sealing layer 1305. The sealing layer 1305 is disposed on or in the structural section 220 and configured to seal the at least one stiffening recess in a fluid-tight manner. The sealing layer 1305 can be formed by potting or overmolding the structural section 220 with a biocompatible plastic. The sealing layer 1305 seals the structural section 220 in a fluid-tight manner so that the blood flow can be drawn into the inlet section 230 and pumped through the line device 105, along the structural section 220, into the outlet unit and thus into the aorta without loss. The sealing layer 1305 is made of a plastic that is still soft enough after curing to withstand the movements of the line device 105 during operation of the cardiac support system, e.g. polyurethane or silicone. When the cardiac support system, in particular the line device 105, is pushed through the aortic arch during a surgical procedure, cracking of the plastic of the sealing layer 1305 is avoided by the use of an appropriately selected material of the sealing layer 1305. The applied plastic can also be used as a fastener and as mechanical protection for a sensor cable integrated into the line device.

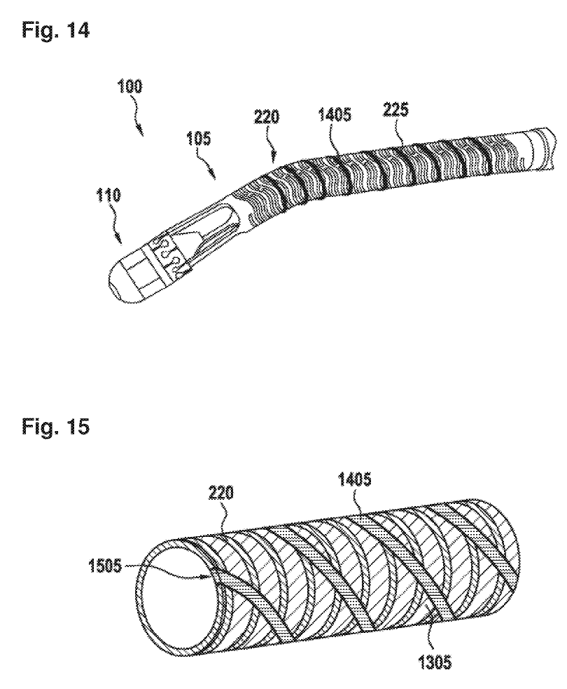

FIG. 14 shows a schematic illustration of a part of a cardiac support system 100 according to a design example. The cardiac support system 100 is the same as or similar to the cardiac support system of one of the figures described above. The figure shows a side view of the section of the cardiac support system 100 with the head unit 110 and the line device 105. As an example, the head unit 110 here is configured as a sensor assembly. From the head unit 110, a sensor cable 1405 is guided along the inner side of the line device 105 on the inside from the proximal attachment section 210 through the inlet section 230 and to the outer side of the line device 105 through the stiffening recess 225 closest to the inlet section 230 resting on the structural section 220. The sensor cable 1405 continues along the longitudinal axis of the line device 105 wound helically around the structural section 220. Leaving a continuous helix makes it possible to secure the sensor cable 1405, which enables an electrical data and power connection from the sensor tip, i.e. the head unit 110, to the pump in a section of the cardiac support system disposed downstream of the outlet unit, in a break proof manner, for example by gluing, overmolding, or potting the sensor cable 1405. The regular movement of the line device 105 during operation of the cardiac support system 100, due to the pulse and movements of the patient, is thus not passed on to the cable.

FIG. 15 shows a schematic illustration of a structural section 220 of a line device according to a design example in a side view. The line device is the same as or similar to the line device of one of the figures described above. The structural section 220 here is made of Nitinol as an example, and comprises a continuous helix 1505 to which the sensor cable 1405 is fastened. The stiffening recesses of the structural section 220 are sealed with the sealing layer 1305. As an example, the sensor cable 1405 is configured as a flexible thin-film substrate and is largely decoupled from mechanical stresses by the continuous helix 1505 to prevent tensile stresses and cable breakage. The helix 1505 is slightly wider than the sensor cable 1405, e.g. by approx. 0.5 to 1 millimeter. When the structural section 220 bends, the spacing of the stiffening recesses on the outside of the bend of the structural section 220 increases, while the stiffening recesses on the inside of the bend are pushed together. The continuous helix 1505 is not or only slightly deformed by the bending of the structural section 220 and thus provides mechanical protection for the sensor cable 1405.

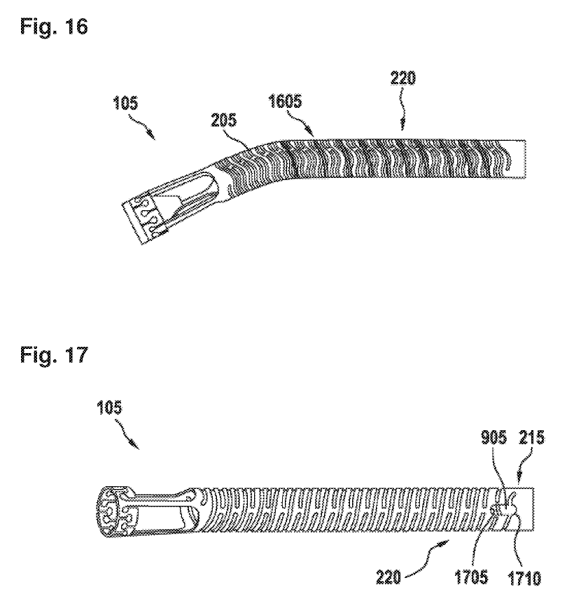

FIG. 16 shows a schematic illustration of a line device 105 according to a design example. The line device 105 is the same as or similar to the line device of one of the figures described above. According to the design example shown here, the line device 105 comprises a cable groove 1605, wherein the cable groove 1605 is formed to guide a cable along the main body 205, in particular wherein the cable groove 1605 is configured to extend helically around the structural section 220. The sensor cable, for example, can be accommodated in the cable groove 1605 as described with reference to the previous FIG. 14 and FIG. 15. In this case, the cable groove 1605 is formed to at least partially accommodate the sensor cable in order to provide additional mechanical protection for the sensor cable. The figure therefore shows a laser-cut design example of the line device 105 with an integrated cable groove 1605 to provide additional mechanical protection for an integrated cable.

FIG. 17 shows a schematic illustration of a line device 105 according to a design example. The line device 105 is the same as or similar to the line device 105 of FIG. 10, with the exception of the lead-out opening 905. In the design example shown here, part of the lead-out opening 905 is disposed in the structural section 220 and the other part is disposed in the distal attachment section 215. Here too, the lead-out opening 905 for leading out the guide wire is cut into the main body of the line device 105 as an elongated recess. The lead-out opening is furthermore chamfered, for example, on both the inner side of the line device 105 and the outer side of the line device 105 and thus has an inner diameter chamfer 1705 and an outer diameter chamfer 1710.

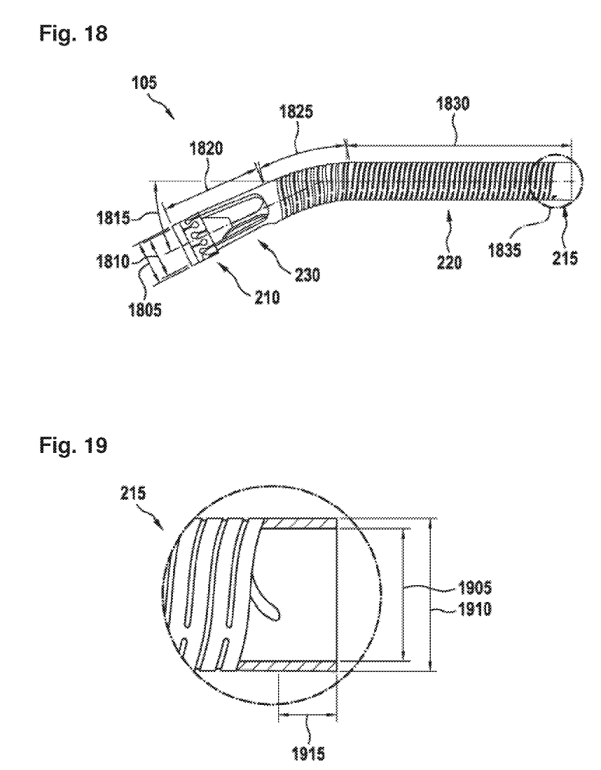

FIG. 18 shows a schematic illustration of a line device 105 according to a design example. The shown line device 105 is the same as or similar to the line device 105 described with reference to FIG. 10, whereby the figure also includes labels to show exemplary dimensions of the line device 105. On the proximal attachment section 210, the line device 105 has an inner diameter of 6.5 millimeters indicated by the label 1805. The outer diameter in this region indicated by the label 1810 is 7 millimeters. The angle of the bending point indicated by the label 1815 is 26 degrees. The label 1820 indicates a length of 15 millimeters of a region of the line device 105, which comprises the proximal attachment section 210 and the inlet section 230, and a region of the structural section 220 with the stiffening recess closest to the inlet section 230. An adjoining, bent section of the structural section 220 that extends obliquely to the longitudinal axis of the line device 105 has a length of 14 millimeters, as indicated by the label 1825. The adjacent section of the line device 105 provided with the label 1830 comprises a remaining region of the structural section 220 and the distal attachment section 215, whereas the circular label 1835 indicates the region of the line device 105 with the distal attachment section 215, which is described in the following FIG. 19.

FIG. 19 shows a schematic illustration of a part of a line device according to a design example. The figure shows the end section of the line device with the distal attachment section 215 marked in the previous FIG. 18 with labels illustrating exemplary dimensions. On the distal attachment section 215, the line device has an inner diameter of 5.5 millimeters indicated by the label 1905. The outer diameter indicated by the label 1910 in this region is 6 millimeters. The label 1915 indicates a section of the distal attachment section 215 with a length of 2.40 millimeters.

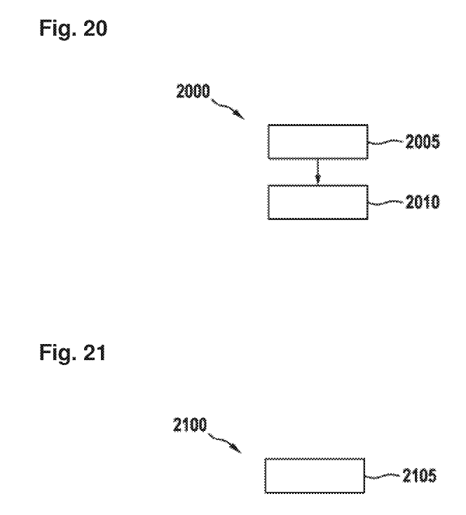

FIG. 20 shows a flow diagram of a method 2000 for producing a line device for conducting a blood flow for a cardiac support system according to a design example. The method 2000 comprises a forming step 2005 and a heat treating step 2010. In the forming step 2005, a main body is formed from a semi-finished product made of a shape memory material. The main body comprises a proximal or first attachment section at a first end for attaching the line device to a head unit of the cardiac support system and comprises a distal or second attachment section at a second end for attaching the line device to an outlet unit of the cardiac support system. The attachment sections are formed to be connectable in a form-locking and/or force-locking manner. The main body further comprises a structural section having at least one stiffening recess between the attachment sections, wherein the at least one stiffening recess is formed to change the stiffness of the main body. The main body also optionally comprises an inlet section between the structural section and the proximal or first attachment section, wherein the inlet section is formed to allow initiation of the blood flow. In the heat treating step 2010, the main body formed in the forming step 2005 is heat treated to emboss a predetermined shape on the main body.

FIG. 21 shows a flow diagram of a method 2100 for assembling a cardiac support system according to a design example. By carrying out the method 2100 for assembling, a cardiac support system that is the same as or similar to the cardiac support system of one of the figures described above can be assembled. The method 2100 comprises a step 2105 of producing a form-locking and/or force-locking connection of the head unit and the outlet unit to the line device to assemble the cardiac support system.

If a design example includes an “and/or” conjunction between a first feature and a second feature, this should be read to mean that the design example according to one embodiment comprises both the first feature and the second feature and, according to another embodiment, comprises either only the first feature or only the second feature.

1. Line device (105) for conducting a blood flow for a cardiac support system (100), wherein the line device (105) has the following features:
a main body (205), wherein the main body (205) comprises a first attachment section (210) at a first end for attaching the line device (105) to a head unit (110) of the cardiac support system (100) and comprises a second attachment section (215) at a second end for attaching the line device (105) to an outlet unit (115) of the cardiac support system (100), wherein the attachment sections (210, 215) are formed to be connectable in a form-locking and/or force-locking manner, wherein the main body (205) comprises a structural section (220) having at least one stiffening recess (225) between the attachment sections (210, 215), wherein the at least one stiffening recess (225) is formed to change the stiffness of the main body (205).
